(19) United States
(12) Patent Application Publication
(10) Pub. No.: US 2025/0109088 A1
(43) Pub. Date: Apr. 3, 2025

(54) PROCESS FOR THE PRODUCTION OF GLYCOLIC ACID

(71) Applicant: Avantium Knowledge Centre B.V., Amsterdam (NL)

(72) Inventors: Gerardus Johannes Maria Gruter, Amsterdam (NL); Eric Schuler, Amsterdam (NL); Shiju Nirappurackal Raveendran, Amsterdam (NL)

(21) Appl. No.: 18/834,488

(22) PCT Filed: Mar. 14, 2023
(86) PCT No.: PCT/EP2023/056409

§ 371 Date: Jul. 30, 2024

(30) Foreign Application Priority Data
Mar. 15, 2022 (EP) 22162185.7

(51) Int. Cl. C07C 51/377; B01J 23/62

(52) U.S. Cl. CPC C07C 51/377; B01J 23/626

(57) ABSTRACT

A process for the production of glycolic acid may include subjecting an aqueous oxalic acid solution to a hydrogenation reaction in the presence of hydrogen and a metal containing hydrogenation catalyst. The process may be
a continuous flow process in a fixed bed reactor and
the aqueous oxalic acid solution and a hydrogen gas stream are fed to the fixed bed reactor, the aqueous oxalic acid solution in the feed having an oxalic acid concentration up to 100% saturation at the feed temperature.

The reactor may include a hydrogenation catalyst bed, the catalyst being a supported metal containing hydrogenation catalyst and
the hydrogenation reaction is performed at a temperature selected from the range of from equal to and higher than 40° C. up to equal to and lower than 85° C., at a hydrogen pressure selected from the range of from equal to and higher than 10 bar H₂ up to equal to and lower than 150 bar H₂.

### FIELD OF THE INVENTION

The present invention relates to a process for the production of glycolic acid comprising hydrogenation of oxalic acid.

### BACKGROUND OF THE INVENTION

In view of climate change issues, lots of initiatives are developing to decrease the amount of greenhouse gases in the atmosphere. The focus on capturing CO₂ is increasing, wherein CO₂ is captured mostly from large point sources, such as chemical or power plants, industries with significant CO₂ emissions (such as steelmaking), natural gas processing, the production of hydrogen from fossil fuels, etcetera. After capture, the next steps are CO₂ storage (CCS) and/or utilization (CCU). The significant difference between storage and utilization is that CCS technologies store CO₂ underground so that it cannot re-enter the atmosphere, whereas CCU technologies use CO₂ to convert it to more valuable products, such as plastics or biofuels. Notably, while today the traditional fossil sources of energy production can be replaced by net-zero emission methods (at least in theory), this is not the case for the production of materials, like plastics or concrete. Thus, using CO₂ for the production of materials will be vital for ensuring a lower impact on natural resources and is one of the few options for realizing negative emissions (provided that at least non-fossil CO₂ and renewable energy is used).

For example, CO₂ can be used to produce oxalic acid (HOOCCOOH), and in turn oxalic acid can be hydrogenated to produce for example glycolic acid (HOCH₂COOH; GA) and mono ethylene glycol (HOCH₂CH₂OH; MEG), which are valuable chemicals for a number of different applications, such as for the production of plastics, and for GA as a preservative in food processing, as a skin care agent in cosmetics, and more.

However, although hydrogenation of carboxylic acids is an important organic reaction for the synthesis of useful and valuable chemicals, it is a chemically difficult reaction due to the low reactivity of the carboxy group and the acidic property. This requires for example rational design of catalysts. See: Tamura, M. et al. Recent Developments of Heterogeneous Catalysts for Hydrogenation of Carboxylic Acids to Their Corresponding Alcohols. Asian J. Org. Chem. 2020, 9 (2), 126-143.

Thus, the direct reduction of carboxylic acids requires severe conditions. However, high temperatures cannot be used, as oxalic acid starts to decompose above 130° C., leading to CO₂ and formate, and further to methane under hydrogenation conditions. As a result, there are only a few disclosures in the art about this particular reaction.

The catalytic hydrogenation of oxalic acid by a ruthenium-carbon catalyst was reported by Santos et al. (Reaction Kinetics, Mechanism and Catalysis (2020) 131:139-151). They investigated in batch mode the reduction of oxalic acid in the presence of a 5 wt. % Ru/C microporous catalyst in a slurry reactor, at a pressure of 80 bar and a temperature range of 120-150° C., under continuous gas flow, with an operation time of 7 hours. They observed the formation of glycolic acid, acetic acid, ethylene glycol, and volatile compounds. Conversions of oxalic acid of above 90% were reached, with highest operating selectivities of 63% (120° C.) for glycolic acid, 16% (130° C.) for ethylene glycol and 87% (150° C.) for volatile products, respectively.

WO2017134139 discloses a method of preparing glycolic acid and/or ethylene glycol, the method at least comprising the steps of: (a) providing an aqueous oxalic acid containing stream having a molar ratio of water/oxalic acid of above 5.0; (b) subjecting the aqueous oxalic acid containing stream provided in step (a) to hydrogenation in the presence of a hydrogenation metal catalyst and hydrogen, thereby obtaining a glycolic acid containing stream; and (c) optionally subjecting the glycolic acid containing stream obtained in step (b) to hydrogenation in the presence of a hydrogenation metal catalyst and hydrogen, thereby obtaining an ethylene glycol (HOCH₂CH₂OH) containing stream. All the examples of WO2017134139 work in batch mode. The highest selectivity for glycolic acid reported in the batch process of WO2017134139 is 76% at a conversion of oxalic acid of 80%, produced after a reaction time of 4 hours at 100° C. and a H₂ pressure of 100-120 bar.

From the Santos publication and WO2017134139, respectively, it can be learned that at relatively high temperature (120° C.) and an operation time of 7 hours, the conversion of oxalic acid is relatively high (91%), which however is at the expense of the selectivity towards glycolic acid (only 63%); whereas at lower temperature (100° C.) and a reaction time of 4 hours, the selectivity towards glycolic acid is higher (76%), but then the conversion of oxalic acid decreases (80%).

There is still a need for a process for the conversion of oxalic acid into glycolic acid featuring both high conversion of the oxalic acid starting material and a high yield of the desired glycolic acid product. Notably, the systems used in the Santos processes with residence times of 4-7 hours are not practical. It would be particularly advantageous to provide an industrially applicable, continuous process for the hydrogenation of oxalic acid to selectively produce glycolic acid.

### SUMMARY OF THE INVENTION

Accordingly, the present invention provides a process for the production of glycolic acid comprising subjecting an aqueous oxalic acid solution to a hydrogenation reaction in the presence of hydrogen and a metal containing hydrogenation catalyst,
wherein the process is a continuous flow process in a fixed bed; and
wherein the aqueous oxalic acid solution is potassium free; and
wherein the aqueous oxalic acid solution and a hydrogen gas stream are fed to the fixed bed reactor, the aqueous oxalic acid solution in the feed having an oxalic acid concentration up to 100% saturation at the feed temperature; and
wherein the reactor comprises a hydrogenation catalyst bed, the catalyst being a supported metal containing hydrogenation catalyst with a total metal loading of from equal to and higher than 2.0 wt % up to equal to and lower than 20.0 wt %; and
wherein the hydrogenation reaction is performed at a temperature selected from the range of from equal to and higher than 40° C. up to equal to and lower than 85° C., at a hydrogen pressure selected from the range of from equal to and higher than 10 bar H₂ up to equal to and lower than 150 bar H₂, at a residence time of equal to or longer than 5 minutes up to equal to or lower than 1 hour;
to produce glycolic acid with high selectivity of 80% and higher, at a conversion of oxalic acid of 80% to 100%.

Contrary to expectation, in the current continuous flow process high conversion of oxalic acid and high selectivity for glycolic acid were found at temperatures significantly lower than the reaction temperatures of the prior art processes, at shorter reaction times/residence times (commercially attractive time frame).

Advantageously, the novel process opens opportunities for efficient industrial production of glycolic acid from oxalic acid.

### DETAILED DESCRIPTION OF THE INVENTION

The present invention relates to a continuous flow process for the production of glycolic acid from oxalic acid. Continuous flow means the process can run uninterrupted and frequent feedstock charging is not needed, as opposed to batch processes. Until now, only some prior art publications relate to the production of glycolic acid from oxalic acid, and those publications only describe the process in batch mode. A “batch process” refers to a process that involves a sequence of steps followed in a specific order, and which process has a beginning and an end. The present continuous flow process refers to a process defined by a flow of reactants and products (for every step) wherein the process runs for a longer period of time while continuously feeding fresh reactants and continuously removing the product. Batch processing usually requires more energy, costs more money, and takes longer, but it is often considered the safest and most manageable way to process certain compounds. Continuous processing, though efficient and cost-effective, is not always suitable for any chemical reaction.

The presently disclosed continuous flow process is performed in a fixed bed reactor (also called packed bed reactor), which can be any fixed bed reactor known in the art, for example including a reactor being a single cylindrical tube, but also a multitubular reactor, properly filled/packed with (a) suitable catalyst(s). The catalyst bed, often in the form of pellets, is placed in such a way that it does not move with respect to the reactor itself. The reactants, including reactant gases, uniformly flow over the fixed catalyst bed. A preferred type of fixed bed reactor for performing the present process is a trickle bed reactor. Trickle bed reactors comprise a family of reactors in which gas phase reactants react with liquid phase reactants while flowing in downward direction (toward the direction of gravity) over a bed of solid catalyst particles. The gas phase flow may be both in upward or downward direction depending on the type of application. Preferably, in the present process, the gas phase reactant (hydrogen gas) flows in downward direction, together with the liquid phase reactant (aqueous oxalic acid solution).

According to the process, a potassium free aqueous oxalic acid solution and a hydrogen gas stream are fed to a fixed bed reactor, the aqueous oxalic acid solution in the feed having an oxalic acid concentration up to 100% saturation at the feed temperature. The term “saturation” as used herein relates to the solubility of oxalic acid in aqueous solution, i.e. it defines the degree to which oxalic acid is dissolved at the given temperature, as compared to the maximum possible degree. An oxalic acid aqueous solution at 100% saturation means that at that temperature a maximum amount of oxalic acid is dissolved. Examples of oxalic acid aqueous solutions at 100% saturation at different temperatures: 46.9 g/L (5° C.), 57.2 g/L (10° C.), 75.5 g/L (15° C.), 95.5 g/L (20° C.), 118 g/L (25° C.), 139 g/L (30° C.), 178 g/L (35° C.), 217 g/L (40° C.), 261 g/L (45° C.), 315 g/L (50° C.), 376 g/L (55° C.), 426 g/L (60° C.), 548 g/L (65° C.) (see Wikipedia page on oxalic acid: reference A. Apelblat et al (1987), The Journal of Chemical Thermodynamics, volume 19, issue 3, pages 317-320). Suitably, a high concentration of oxalic acid is used, preferably at least an 80% aqueous solution of oxalic acid, however more preferably, the concentration being as high as possible at the feed temperature, i.e. most preferably at 100% saturation.

In weight percentages, preferably the aqueous oxalic acid solution comprises from equal to or higher than 1.0 weight % to equal to or lower than 40 weight % of oxalic acid, preferably equal to or higher than 2.5 weight %, more preferably equal to or higher than 5.0 weight % to equal to or lower than 20 weight % of oxalic acid.

The selected temperature of the oxalic acid aqueous solution in the feed (“feed temperature”) generally is the same temperature as the temperature at which the hydrogenation reaction is performed.

To increase the solubility of oxalic acid in water, certain additives may be used in the aqueous oxalic acid solution, for example alcohols, ethers, etcetera, which are inert under the reaction circumstances. The presence of such additives in the aqueous solution thus has an effect on the saturation, which means that for example a 100% saturated solution comprises more oxalic acid per liter than in the absence of those additives.

Suitably, the aqueous oxalic acid solution that is used as feed may be a recycle stream from the hydrogenation reaction or it may comprise a recycle stream from the hydrogenation reaction. Said recycle stream can therefore comprise one or more hydrogenation (side) products, such as glycolic acid, ethylene glycol, glyoxylic acid, acetic acid, etc., Preferably, the aqueous oxalic acid solution that is used as feed comprises at most 20 weight % in total of such hydrogenation (side) products.

The aqueous oxalic acid solution used as feed does not comprise potassium in any form. The presence of potassium is considered to be poisonous to the activity of catalyst. The term “potassium free” suitably means that the amount of potassium in the aqueous oxalic acid solution is below 0.9 wt % (9000 ppm), preferably below 0.5 wt % and especially below 0.1 wt %.

Suitably, the catalyst in the fixed bed is a supported metal containing hydrogenation catalyst with a total metal loading of from equal to and higher than 2.0 wt % up to equal to and lower than 20.0 wt %, and preferably from equal to and higher than 3.0 wt % up to equal to and lower than 15.0 wt %, more particularly up to equal and lower than 12.0 wt %.

The hydrogenation catalyst preferably contains one or more metals selected from group A metals: platinum, nickel, copper, ruthenium, rhodium and iridium, and preferably ruthenium. Further, the hydrogenation catalyst optionally also contains one other metal selected from group B metals: tin, bismuth, palladium, rhenium, gold, and antimony. Preferably, the hydrogenation catalyst contains ruthenium (group A metal), and preferably one group B metal.

A highly preferred hydrogenation catalyst contains ruthenium and tin. Such ruthenium-tin catalyst can advantageously be used to suppress side product formation such as over-reduction products like acetic acid and ethylene glycol at high oxalic acid conversion. The molar 30 ratio of ruthenium and tin in the catalyst from 10:1 to 1:10, preferably 5:1 to 1:5, more preferably 5:2 to 1:4.

A further preferred catalyst for use in the process is a trimetallic catalyst containing ruthenium, platinum and tin. See e.g. S. Taniguchi et al./Applied Catalysis A: General 397 (2011) 171-173. The amounts and molar ratio of ruthenium and tin in the trimetallic catalyst are selected as described above. The amount of platinum is preferably 1 to 5 weight %, more preferably 1.5 to 3 weight % relative to the catalyst support. A highly preferred catalyst for optimal production of glycolic acid from oxalic acid is Ru₁Sn_{0.85}Pt_{0.2}/C, the catalyst having a metal loading 5 wt % of ruthenium, 5 wt % of tin and 2 wt % of platinum. The oxalic acid conversion rate is increased when platinum is also present in the catalyst.

Advantageously, no chloride is present in the catalyst used in the present process, which is considered important for catalyst stability.

Preferably, the catalyst used in the process according to the present disclosure is calcined (pre-treated) prior to use at a temperature selected from 200° C. to 450° C.

The catalyst support can be selected from any support material that does not interfere with the current hydrogenation reaction. Preferably, the hydrogenation catalyst is supported on a carrier selected from carbon, silicon carbide, MAX-Phase (Ti₂Al₂C), TiO₂ and ZrO₂, more preferably carbon or MAX-Phase, most preferably carbon.

According to the present invention, reaction conditions were found that are very useful for a continuous flow process for the selective production of glycolic acid at relatively low temperatures, with high oxalic acid conversion. At temperatures from equal to and higher than 40° C., preferably from equal to and higher than 50° C., up to equal to and lower than 85° C., and within a commercially attractive time frame, advantageously a glycolic acid selectivity of particularly at least 80% could be obtained at a conversion of oxalic acid of from equal to and higher than 80% up to and including 100%. In certain advantageous embodiments, the temperature is from equal to and higher than 60° C. up to equal to and lower than 85° C.

The commercially attractive time frame in which the present continuous flow process takes place, is expressed in terms of “residence time”. The term “residence time” defines the average length of time that the feed, or specifically a molecule in the feed, remains inside the reactor, meaning herein inside the part that contains the catalyst bed. According to the process of the present invention, a residence time of equal to or longer than 5 minutes up to equal to or less than 2 hours is required. Preferably, the residence time is equal to or longer than 10 minutes up to equal to or less than 1 hour. For flow reactors suitable for the present hydrogenation reaction, the residence time may be varied by varying factors like the reactor volume, the feed flow rate (both of the oxalic acid solution and of the hydrogen gas), the length of the catalyst bed, etc., When compared to continuous flow processes, in a batch process, the residence time can be related to reaction time, i.e. how long a container is held at a specific temperature in the batch process.

According to general rules in chemistry as laid down in the Arrhenius Equation, as a rule of thumb, the rate of a reaction roughly doubles (or halves) for every 10° C. higher (or lower, respectively). Taking the prior art example of WO2017134139, giving a selectivity of 76% of glycolic acid at a conversion of oxalic acid of 80%, produced after a reaction time of 4 hours at 100° C., these results suggest that performing the reaction at lower temperature will certainly lead to lower conversions (at the same 4 hours of reaction time). As performing the reaction at 10° C. lower typically halves the reaction rate, a reaction according to the prior art batch process WO2017134139 performed at 50° C. is expected to give a 2×2×2×2×2=32 (5 times halved) lower reaction rate than at 100° C. in the same reaction time. In other words, the reaction of WO2017134139 performed at 50° C. would only give an estimated 2-3% conversion after 4 hours. As a consequence, in order to come to a result of 60 to 100% conversion at 50° C., the batch process of WO2017134139 should be run for at least 80 to 200 hours, which evidently is commercially unattractive.

In the process of the invention, it was found that increasing the feed flow does not affect the selectivity towards glycolic acid. If the feed flow is very high, resulting in very short residence times, only the conversion of oxalic acid does not run until completion, but still selectively glycolic acid is formed. The person skilled in the art will understand how to balance the feed flow at a certain temperature against the most appropriate residence time. At a relatively high temperature, shorter residence times (and thus higher feed flow) will be required to maximize both the conversion of oxalic acid and selectivity towards glycolic acid. At higher temperatures (in particular above about 95° C.), ethylene glycol may start to form as a side product before full conversion of oxalic acid.

The current process is performed at a hydrogen pressure selected from the range of from equal to and higher than 10 bar H₂ up to equal to and lower than 150 bar H₂, preferably at 20 to 100 bar H₂.

The product stream of the continuous flow process according to the present disclosure may be continuously removed from the process and directly subjected to further processing if deemed necessary, but also the product stream may be (partly) recycled before the desired glycolic acid is separated from the solution.

Oxalic acid is a corrosive substance. Stainless steel reactors are therefore not ideal for the present process. Advantageously, therefore, the present hydrogenation reaction is performed in a reactor with non-metallic or inert liners, such as Teflon, glass, PVC, titanium or Hastelloy.

A suitable and advantageous way to perform the process of the invention comprises subjecting a potassium free aqueous oxalic acid solution to a hydrogenation reaction in the presence of hydrogen and a metal containing hydrogenation catalyst; wherein the process is a continuous flow process in a trickle flow Hastelloy reactor; wherein an aqueous 5-15% oxalic acid solution and a hydrogen gas stream are fed to the top of the reactor; wherein the reactor contains a Ru₁Sn_{0.85}Pt_{0.2}/C catalyst bed, the catalyst preferably having a metal loading 5 wt % of ruthenium, 4.7 wt % of tin and 2 wt % of platinum; wherein the hydrogenation reaction is performed at a temperature selected from the range of from equal to and higher than 50° C. up to equal to and lower than 85° C., at a hydrogen pressure selected from the range of from equal to and higher than 50 bar H₂ up to equal to and lower than 100 bar H₂, at a residence time of equal to or longer than 10 minutes up to equal to or less than 45 minutes; to produce glycolic acid with high selectivity of 90% and higher, at a conversion of oxalic acid of 90% to 100%.

### BRIEF DESCRIPTION OF THE DRAWING

FIG. 1. Results of QCS experiments with RuSn (5 wt. %/5.9 wt) catalysts on different supports. Conversion (A), Selectivity (B) and Carbon Balance (C) data were obtained by liquid chromatography (LC). Conditions during reactions: Temperature=75° C., Pressure=80 bar, Substrate=oxalic acid (5 wt. %) in demineralized water (2 ml), Catalyst/Support Loading=50 mg, Reaction time=2, 4 and 6 hours, respectively.

FIG. 2. Results of SFU experiment of oxalic acid reduction for different temperatures. Conversion (A), Selectivity (B) and Carbon Balance (C) data as obtained by liquid chromatography (LC). Conditions during reactions: Temperature=50-120° C., Pressure=60 bar, Feed=oxalic acid (5 wt. %) in water, Flow of Feed=0.1 ml min⁻¹ (corresponding to a residence time of 18 minutes for full liquid conditions), Flow of Gas (H₂)=200 ml min⁻¹. In this reaction Ru₁Sn_{2.3}/C (calcined) was used as a catalyst.

FIG. 3. Results of 100 hours SFU experiment of reduction of oxalic acid produced by electrocatalytic CO₂ reduction. Conversion (A), Selectivity (B) and Carbon Balance (C) data as obtained by liquid chromatography (LC). Conditions during reactions: Temperature=50° C., Pressure=60 bar, Feed=Oxalic Acid (2.37 wt. %) in water, Flow of Feed=0.1 ml min⁻¹, Flow of Gas (H₂)=200 ml min⁻¹. Catalyst 9.76 wt. % Ru/C (0.5 wt. % moisture; Jonhson Mattey).

FIG. 4. Results of 100 hours SFU experiment of reduction of commercial oxalic acid. Conversion (A), Selectivity (B) and Carbon Balance (C) data as obtained by liquid chromatography (LC). Conditions during reactions: Temperature=50° C., Pressure=60 bar, Feed=Oxalic Acid (2.37 wt. %) in water, Flow of Feed=0.1 ml min⁻¹, Flow of Gas (H₂)=200 ml min⁻¹. Catalyst 9.76 wt. % Ru/C (0.5 wt. % moisture; Jonhson Mattey).

The invention is further illustrated by the following non-limiting examples.

### EXAMPLES

### List of Abbreviations

AA=acetic acid
EG=(mono) ethylene glycol
GA=glycolic acid
GlyA=glyoxylic acid
MEG=(mono) ethylene glycol
OA=oxalic acid
QCS=Quick Catalytic Screening
SFU=Single Flow Unit

### Experimental

### Materials and Reagents

Oxalic acid (C₂H₂O₄) was obtained from Sigma-Aldrich®), dried and stored in a dry environment. All water used during this work was filtered using a Millipore system.

All commercial catalysts are listed in the table below and were purchased directly from the manufacturer. All catalysts were stored in a dry environment.

| Catalyst | Type | Lot | % H2O | Supplier |
|---|---|---|---|---|
| 5 wt. % Ru/C | D101023-5 | C4198 | 57.15 | Johnson Matthey |
| 5 wt. % Ru/C | | F14X032 | 57.1 | Alfa-Aesar |
| 5 wt. % | Degussa | 10714KY | — | Sigma Aldrich |
| Ru/Al2O3 | H213R/D | | | |
| 5 wt. % Pd/C | A405028-5 | C8738 | 58.37 | Johnson Matthey |
| 5 wt. % Ru/C | D101002-5 | C-11266 | | Johnson Matthey |
| 5 wt. % Ru/C | 5R600 | C-12052 | | Johnson Matthey |
| 7.5 wt. % Ru/C | D102023-7.5 | C-11106 | | Johnson Matthey |
| 10 wt. % Ru/C | D101023-10 | C-11309 | | Johnson Matthey |
| 5 wt. % | D302011-5 | C-12053 | | Johnson Matthey |
| Ru/Al2O3 | | | | |
| 5 wt. % Ru/C | Type 619 | M11099 | | Alfa-Aesar |
| 5 wt. % Ru/C | | H30Z004 | | Alfa-Aesar |
| 10 wt. % Ru/C | | C09Y010 | | Alfa-Aesar |
| 7.5 wt. % Ru/C | | C-12136 | | Johnson Matthey |
| 7.5 wt. % Ru/C | | C-1213 | | Johnson Matthey |

Another catalyst that was used was 9.76 wt. % Ruthenium on Carbon, supplied by Johnson Matthey (ID: 110005, LOT M17160). The catalyst contained 0.5% moisture.

All catalyst support materials: Carbon (NORIT SX 1 G), Ti₂Al₂C (MAX-Phase), Titania (TiO₂), Alumina (γ-Al₂O₃) and Zirconia (ZrO₂), were obtained from commercial suppliers (Sigma-Aldrich®), and dried and stored in a dry environment.

### Catalyst Reduction and Preparation

Catalyst extrudates were crushed with a ceramic mortar and sieved to obtain a mesh size between 105-200 μm. Catalyst particles were transferred to a ceramic crucible and placed in a tubular furnace for reduction. In the furnace, the particles were treated with a gas mixture of 7% H₂ in N₂ with a flow rate of 100 ml min⁻¹. The temperature inside the furnace was increased with a ramp of 10° C. min⁻¹ until it reached 300-450° C. This temperature was kept constant for 180 minutes. Upon completion of the reduction procedure, the temperature was slowly decreased, and the 7% H₂/N₂ mixture was flushed out with a flow of pure nitrogen.

### Catalyst Screening

We used QCS reactions to compare the performance of the synthesized catalyst for the reduction of oxalic acid (using a Batchington reactor for quick catalyst screening (QCS) developed by Avantium Technologies). For these reactions tantalum reactors were used instead of stainless steel reactors. These reactors were equipped with Teflon liners that were inserted in the reactors. Pre-reduced catalyst (see section Catalyst reduction and preparation) (50±2.5 mg) was loaded into individual reactors, after which the reactors were stored for 16 hours. The reactors were closed from the top to prevent air from leaking in and oxidizing the catalysts during the reactor storing step. After 16 hours stirring beans were added to the individual reactors. 2 ml of a stock solution of oxalic acid (5 wt. %) in demineralized water was transferred to the individual reactors with a Gilson Pipetman Concept, motorized air-displacement pipette. Septa were placed on the individual reactors. The reactor lid was attached in a ‘criss-cross’ tightening sequence starting from the middle. A torque wrench (5 N m) was used to create an equal distribution of force among the different screws. The reactors were flushed 3 times with N₂ (10 bar) and 3 times with H₂ (10 bar), after which they were put under ˜79-bar pressure of H₂ in a pressurizer. The temperature during QCS was not actively measured, instead an equilibration procedure was performed in advance of an experiment to determine the required settings to maintain 75° C. during the reaction. A stirring speed of 800 rpm was used during the experiment. To stop the reaction the reactors were removed from the QCS unit and immediately cooled down in an ice bath to stop the reaction from continuing. The QCS lid was removed by detaching the screws in a similar ‘criss-cross’ pattern as was used during the initial attaching sequence. Gas could be heard escaping from the reactors as the lid was detached. The catalyst was removed from the reaction mixture by transferring the liquid part of the mixture through a syringe M-filter, which blocked solid particles from being transferred.

Ru—Sn catalysts (5 wt. %/5.9 wt. %, corresponding to molar ratio of Ru:Sn of 1:1) on different supports (Carbon, TiO₂, ZrO₂, Al₂O₃ and Ti₃(Al_{0.8}Sn_{0.2})C₂ MAX-phase) were prepared via wet-impregnation. Before testing, the catalysts were reduced ex-situ at 350-500° C. in a hydrogen atmosphere.

QCS experiments were performed with ruthenium-tin (5 wt. %/5.9 wt) catalysts on the different supports. Conversion (A), Selectivity (B) and Carbon Balance (C) data were obtained by liquid chromatography (LC). Conditions during reactions: Temperature=75° C., Pressure=80 bar H₂, Substrate=oxalic acid (5 wt. %) in demineralized water (2 ml), Catalyst/Support Loading=50 mg (pre-reduced in H₂ ex-situ at 350° C. for 3 h and in-situ at 200° C. for 2 h), Stir rate 800 rpm, Time=2, 4 and 6 hours.

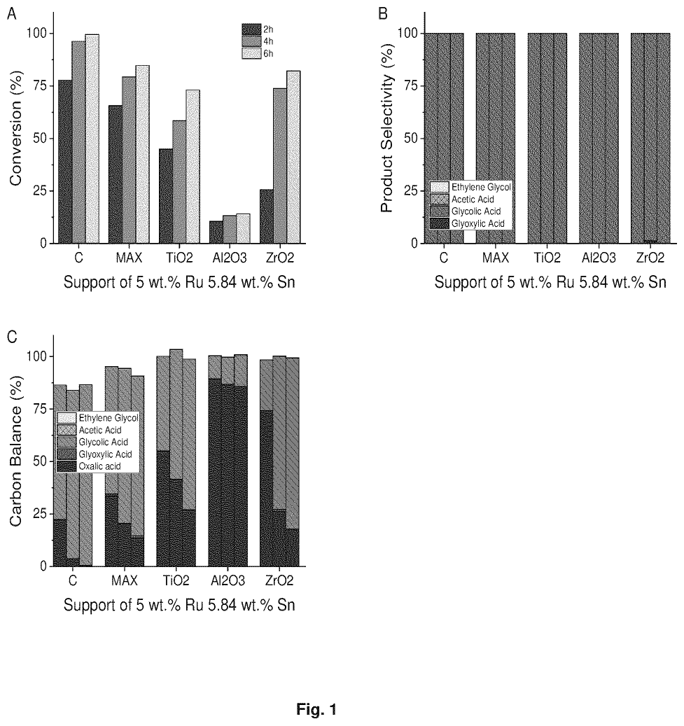

FIG. 1 shows results of QCS experiments on the different supports after 2, 4 and 6 hrs. The percentage of conversion was measured (A), product selectivity (B), and carbon balance (C) of the reaction, wherein amounts of EG, AA, GA, GlyA, and OA were determined.

### Single Flow Unit (SFU) Components

Flow chemistry experiments were performed in a trickle bed reactor system. This unit can be divided into different sub-sections. The gas flow was controlled by 2 mass flow controllers (MFC's) for both nitrogen and hydrogen. The Nitrogen MFC was capable of a flow up to 1000 ml min⁻¹, whereas the Hydrogen MFC was able to go up to 200 ml min⁻¹. The pressure in the reactor was controlled by a pressure indicator, which puts pressure on a back pressure regulator located after the reactor to regulate the pressure inside the system. The feed section consisted of a Jasco HPLC pump. The liquid feed flow rate range was between 0.1 and 5 ml min⁻¹, which was transferred separately from the gas flow to the reactor.

The reactor used for the experiments was a 30 cm long reactor tube, with a diameter of 4.6 mm. The reactor volume of this reactor was 5 ml, of which 1.82 ml was within the isothermal zone of the system and therefore allowed to be loaded with a catalyst. For full liquid flow in plug-flow mode the max residence time for this system will be 18 minutes with a liquid flow rate of 0.1 mL per minute. In trickle flow operation the residence time will be somewhat lower (less liquid present in the reactor).

This system had space for 8 separate effluent sample vials, which collected effluent that had passed the reactor and directed to the sample vial through a selector valve system. The sample vials had 2 needles inserted, one input needle from the selector valve and one output needle to release pressure or overflow feed towards a “Flush” jerry can. The selector valve itself had 16 channels, of which 8 channels led towards the sample vials and 8 channels that went through a manifold towards a “Waste” jerry can.

The system was provided with a heating and stirring system for the feed solution which allowed for heating and stirring of the feed solution to create a more homogeneously distributed feed solution.

### Reactor Materials

As oxalic acid is a good complexing agent, it can potentially leach metals from the reactor components or the catalyst. To get an idea for leaching, we analysed the reaction solution after reaction of 25 wt % OA in water in a Hastelloy reactor for 2 hours at 65° C. with ICP-OES. We observed strong leaching of chrome, iron, and nickel but no leaching of ruthenium from the catalyst. To study the effect of these metals in the solutions we added three times the amount found in the leaching experiments of Cr, Fe, Ni to the starting solution. We noticed inhibition of the initial reaction rate by 10-15%. To prevent leaching, we tested non-metallic liners for the Hastelloy reactor and avoided any metal parts contacting the reaction solution. We compared the performance of the reactor with and without Teflon and glass liners and found better catalyst stability with liners. We performed the reaction six times consecutively with the same solvent to amplify the potential effect of leaching. For each of the six reactions, we added fresh reactant solution but kept the catalyst in the reactor. The Teflon liners worked best to preserve the catalytic activity as the high over-reduction to ethylene glycol. Metal ions from the reactor corrosion at ppm levels (Ni, Cr, Mo) might be inhibiting the catalyst performance and only Teflon liners provide adequate protection.

### Reactor Loading

Single flow reactions used a 30 cm long stainless steel reactor. Equilibration experiments were performed before this study on this reactor to determine that the isothermal zone lies between 9 and 20 cm from the bottom up. The internal diameter of the reactor was 4.6 mm, which corresponds to a reactor volume of 5 ml (or 1.82 ml for the isothermal zone). For each reactor loading, the maximum volume (1.82 ml) of catalyst was used to fill up the isothermal zone of the reactor. The catalyst was reduced ex-situ at 350° C. in a hydrogen atmosphere, packed into the reactor bed and pre-reduced in-situ in the flow reactor at 200° C. in a hydrogen atmosphere. The remaining part of the reactor was filled up with layers of Silica Carbide (SIC) and Quartz wool

### Reactor Attaching

The stainless steel reactor was equipped with Swagelok fittings. Through standard Swagelok attaching procedure, the reactor was attached to the SFU. Swagelok fittings are the choice of fitting here because of their ability to hold high pressures while remaining leak-free.

### Analysis

Liquid chromatography was performed as follows. Samples were prepared by diluting stock solutions to concentrations of 0.67 mg stock mL⁻¹ in demineralized water. An Agilent Technologies 1260 Infinity II was used to measure the concentration of oxalic acid, glycolic acid, glyoxylic acid, acetic acid, and ethylene glycol. To confirm the results by a second method we used quantitative liquid phase IR measurements in random order. The results for both methods matched.

### Examples

The system used for these reactions is a trickle-bed reactor system, which allows unlimited continuous operation and automated collection of 8 samples per experiment without interruption. Jasco HPLC pumps create a reactant feed flow, while mass flow controllers (MFCs) control gas (H₂ or N₂) flow towards the reactor. On top of the reactor, both the feed and gas flow merge, which causes a downward movement of the liquid and co-current movement of the gas over the packed catalyst bed.

The effect of the different reaction parameters were studied, such as reaction temperature (50-100° C.), hydrogen pressure (10-60 bar), hydrogen flow (50-200 mL min⁻¹), liquid/gas ratio, residence time, equilibration steps, and in/exclusion of a pre-reduction step and respective temperature. The length and volume of the catalyst bed and the packing thereof, and the OA concentration (5 wt. % OA in water) were kept constant for all experiments.

For each experiment, fresh catalyst was used.

### Control Experiments

(1) Absence of catalyst—Control experiments were performed to exclude any possible reaction of the trickle bed reactor with the reactant feed. These control experiments employed similarly loaded reactors as described in the section “Reactor Loading”. For the control experiments, the catalyst in the isothermal zone of the system was replaced with silica carbide. Different reactant feed flow rates (5 to 0.1 ml min⁻¹) were examined.

Results: Near-zero conversion of oxalic acid was observed towards observable reaction products during these reactions. The observed marginal conversion lies within the margin of error of the analysis method. Liquid chromatography showed only oxalic acid. In conclusion, in the employed system no oxalic acid converts in the absence of any catalyst.

(2) No hydrogen—The influence of hydrogen presence was investigated. The reactor according to the procedure described in the section “Reactor Loading”. For this experiment, a commercial catalyst provided by Johnson Mattey with the following characteristics (9.76 wt. % Ru/C, 0.5 wt. % moisture) was loaded within the isothermal zone of the reactor. Instead of a hydrogen flow, different flows of nitrogen were examined. The reaction conditions during this experiment were similar to conditions of the actual experiments described herein that did yield glycolic acid, the only exception being a nitrogen flow instead of hydrogen.

Results: Similar to the results of the control experiment in the absence of a catalyst, this experiment yielded no reaction products. The conversions were so marginal that they are within the margin of error of the LC analysis, and no conclusions regarding any possible conversion can be derived from these data.

From the controls and pre-experiments, it was concluded that the conversion of oxalic acid requires the presence of a catalyst and pressurized hydrogen.

Note: the by-product ethylene glycol is derived from the reduction of glycolic acid, whilst acetic acid is formed from oxalic acid directly [as for example discussed by Santos et al. (Reaction Kinetics, Mechanism and Catalysis (2020) 131:139-151)].

### Example 1

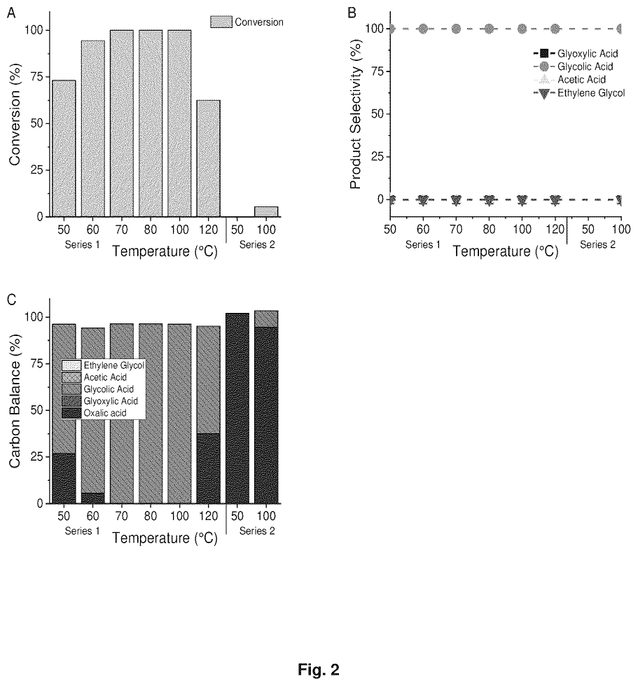

We tested the catalyst Ru1Sn2.3/C [the molar ratio of Ru:Sn of 1:2.3 was in our experiments the most active], containing 5 wt % of ruthenium and 13.7% wt % of tin, in the trickle-bed flow reactor. We examined the catalyst performance in the temperature range 50-120° C. The conversion increased and reached 100% at 70° C. and solely glycolic acid was formed (FIG. 2, “Series 1”). The formation of acetic acid (as previously observed above 70° C. with Ru/C catalyst) and any over-reduction of glycolic acid to ethylene glycol was avoided by the addition of Sn to the catalyst. 100% glycolic acid yield was obtained in the range 70-100° C. As we increased the temperature further to 120° C., the catalyst deactivated and the conversion dropped by 38%. Subsequently, for a following experiment with the used catalyst at for example 50 and 100° C., it appeared that the catalyst had deactivated above the applied high temperatures (see FIG. 2, “Series 2”). Also, at 100° C. the catalyst deactivated at longer reaction times.

### Comparative Example 2. Effect of Potassium

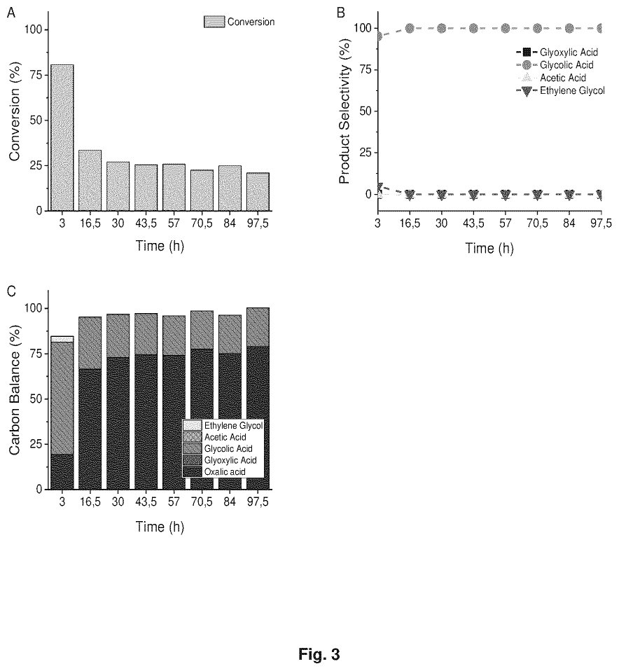

As this work is part of the development of an industrial process from CO₂ to chemicals or polymers, we tested the reaction with the real feed coming from the electrochemical acidification reactor in which the oxalic acid is produced from potassium oxalate. The only difference to the conditions established earlier was a lower concentration of oxalic acid at 2.37 wt. % in water. To prove the stability of the process, we aimed at least at a reaction time of 100 hours in a single uninterrupted reaction. Unfortunately, the conversion dropped drastically already after 16.5 hours (FIG. 3).

To be read in connection with Example 3.

### Example 3

Earlier we had used the same catalyst as used in comparative Example 2 for more than 100 hours without much deactivation, so we attributed the cause to the real feed and tested it for contaminants. It turned out, that not all potassium oxalate was converted to oxalic acid, leaving 9000 ppm of potassium, a known catalyst poison, in the feed.

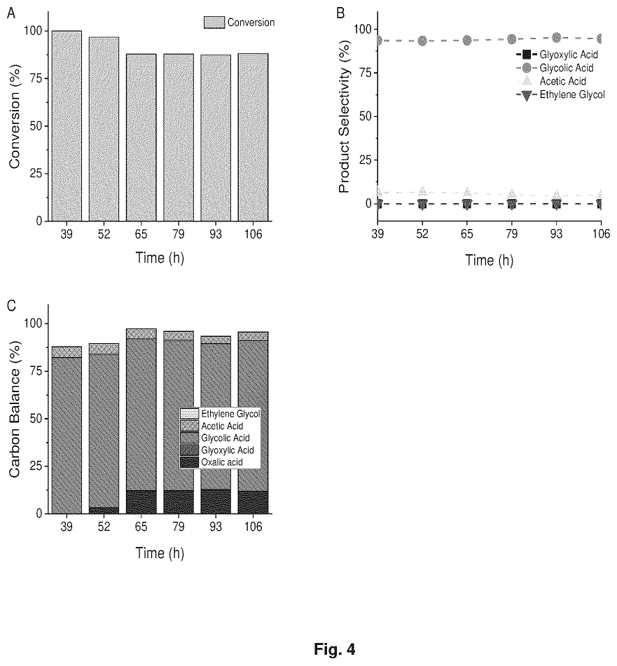

To be able to compare side-by-side with comparative Example 2, we prepared a potassium free solution with the same oxalic acid concentration with which the conversion stabilized at 88% conversion after 56 hours which stayed constant for more than 100 hours (FIG. 4). The conversion could be increased by increasing the temperature, residence time or hydrogen pressure. The selectivity towards glycolic acid was 94.5% but [without Sn in the catalyst] the formation of acetic acid still reached 5.5% (4.3 min, 6.7% max).

1. A process for the production of glycolic acid comprising subjecting an aqueous oxalic acid solution to a hydrogenation reaction in the presence of hydrogen and a metal containing hydrogenation catalyst,
wherein the process is a continuous flow process in a fixed bed reactor; and
wherein the aqueous oxalic acid solution is potassium free; and
wherein the aqueous oxalic acid solution and a hydrogen gas stream are fed to the fixed bed reactor, the aqueous oxalic acid solution in the feed having an oxalic acid concentration up to 100% saturation at the feed temperature; and
wherein the reactor comprises a hydrogenation catalyst bed, the catalyst being a supported metal containing hydrogenation catalyst with a total metal loading of from equal to and higher than 2.0 wt % up to equal to and lower than 20.0 wt %; and
wherein the hydrogenation reaction is performed at a temperature selected from the range of from equal to and higher than 40° C. up to equal to and lower than 85° C., at a hydrogen pressure selected from the range of from equal to and higher than 10 bar H₂ up to equal to and lower than 150 bar H₂, at a residence time of equal to or longer than 5 minutes up to equal to or less than 1 hour; to produce glycolic acid with high selectivity of 80% and higher, at a conversion of oxalic acid of 80% to 100%.

2. The process of claim 1, wherein the reactor is a trickle bed reactor.

3. The process of claim 1, wherein the hydrogenation catalyst contains one or more metals selected from group A metals: platinum, nickel, copper, ruthenium, rhodium and iridium, and optionally one other metal selected from group B metals: tin, bismuth, palladium, rhenium, gold, and antimony.

4. The process of claim 1, wherein the hydrogenation catalyst contains ruthenium as group A metal.

5. The process of claim 4, wherein the hydrogenation catalyst contains ruthenium and tin.

6. The process of claim 5, wherein the molar ratio of ruthenium to tin in the catalyst is from 10:1 to 1:10.

7. The process of claim 1, wherein the catalyst is a trimetallic catalyst containing ruthenium, platinum and tin.

8. The process of claim 1, wherein the hydrogenation catalyst is supported on a carrier selected from carbon, silicon carbide, MAX-Phase (Ti₂Al₂C), TiO₂ and ZrO₂.

9. The process of claim 1, wherein the hydrogenation reaction is performed in a reactor with non-metallic or inert liners.
